(19) United States
(12) Patent Application Publication
(10) Pub. No.: US 2023/0046438 A1
(43) Pub. Date: Feb. 16, 2023

(54) METHOD FOR PREDICTING CELL SPATIAL RELATION BASED ON SINGLE-CELL TRANSCRIPTOME SEQUENCING DATA

(71) Applicant: PEKING UNIVERSITY, Beijing (CN)

(72) Inventors: Zemin ZHANG, Beijing (CN); Xianwen REN, Beijing (CN); Guojie ZHONG, Beijing (CN)

(21) Appl. No.: 17/758,836

(22) PCT Filed: Jan. 14, 2020
(86) PCT No.: PCT/CN2020/072044

§ 371 Date: Jul. 14, 2022

(51) Int. Cl. G16B 5/00; G16B 30/00

(52) U.S. Cl. CPC G16B 5/00; G16B 30/00

(57) ABSTRACT

A method for predicting the cell spatial relation based on single-cell transcriptome sequencing data includes the steps of obtaining a probability matrix P of a cell-cell interaction strength matrix A based on single-cell transcriptome sequencing data; reconstructing, according to the obtained probability matrix P of the cell-cell interaction strength matrix A, a three-dimensional spatial structure in which cells interact with each other; and for each cell in the reconstructed three-dimensional spatial structure in which cells interact with each other, determining the intercellular distance threshold for each cell to interact with h cells on average to obtain an intercellular interaction network. The method requires only the single-cell transcriptome sequencing data to predict the interaction of the cells in three-dimensional space, which breaks the limitation of the existing technology that needs to obtain the spatial relationship of cells through imaging.

### TECHNICAL FIELD

The present disclosure belongs to the technical field of biology, and particularly relates to a method for predicting spatial relations between cells based on single-cell transcriptome sequencing data.

### BACKGROUND

The spatial structure of cells plays an important role in understanding the behavior and functions of cells, and how to obtain the spatial organization of cells in tissues and organs is an important topic in the field of biomedicine.

At present, the method of obtaining the spatial organization of cells is based on experiments, where important genes, proteins or other biomolecules are fluorescently or otherwise labeled and finally the spatial distribution information of cells is obtained by microscopic imaging. In the existing calculation methods, marker genes related to the spatial positions of cells can be determined according to the experimental method described above, and then the marker genes that determine the spatial positions are utilized in combination with single-cell transcriptome sequencing data to map the cells with the transcriptome sequencing data in a known spatial image of cells. There is no calculation method in the prior art that can be used to reconstruct the spatial structures of cells by only using single-cell transcriptome sequencing data without depending on a known spatial image of cells.

In addition, ligand-receptor interactions play an important role in cell interactions and communication. In the existing calculation methods, there is a way in which whether some ligand-receptor pair interaction or the number of ligand-receptor pairs between cell types is significantly greater than those between other cell type pairs can be determined according to the single-cell transcriptome sequencing data; however, reconstruction of cell interactions and the spatial structure of cells at a single-cell level according to the ligand-receptor has not been found.

### SUMMARY

To solve the problems described above, an embodiment of the present disclosure provides a method for predicting spatial relations between cells based on single-cell transcriptome sequencing data, which comprises:

acquiring a probability matrix P of a cell-cell interaction intensity matrix A based on single-cell transcriptome sequencing data and ligand-receptor interactions;

reconstructing a spatial structure (three dimensions by default, a space of two or one dimension or within specific geometry is also applicable) of cell interactions according to the acquired probability matrix P of the cell-cell interaction intensity matrix A; and

determining, for each cell in the reconstructed spatial structure of cell interactions, an intercellular distance threshold where each cell interacts with h cells on average and obtaining an intercellular action network.

Further, a model for reconstructing a three-dimensional spatial structure of cell interactions is as follows:

minimizing an objective function

$${Σ}_{i = 1}^{I} {Σ}_{j = 1}^{I} {p}_{ij} log \frac{{p}_{ij}}{{q}_{ij}} , i \neq j ,$$

such that:

$${q}_{ij} = \frac{1}{{Z}_{q}} \times \frac{1}{1 + {d}_{ij}^{2}} , for i \neq j ,$$

$${d}_{ij}^{2} = \sqrt{{\sum }_{m = 1}^{3} {( {y}_{i}^{m} - {y}_{j}^{m} )}^{2}} for i \neq j ,$$

$${Z}_{q} = {Σ}_{i = 1}^{I} {Σ}_{j = 1}^{I} \frac{1}{1 + {d}_{ij}^{2}} , i \neq j ,$$

wherein, I is a total number of cells;

pᵢⱼ is an interaction intensity between cell i and cell j in the probability matrix P of the cell-cell interaction intensity matrix A;

qᵢⱼ is a probability of cell j being around cell i;

dᵢⱼ is a Euclidean distance between cell i and cell j in a three-dimensional space;

yᵢ^{m} is a coordinate of cell i on axis m;

yⱼ^{m} is a coordinate of cell j on axis m;

Further, the objective function

$${\sum }_{i = 1}^{I} {\sum }_{j = 1}^{I} {p}_{ij} log \frac{{p}_{ij}}{{q}_{ij}} , i \neq j$$

is minimized, cell coordinates are updated using gradient descent, and a gradient direction is calculated for each cell at the present coordinates:

$$\frac{δ C}{{δ y}_{i}} = 4 {\sum }_{j} ( {p}_{ij} - {q}_{ij} ) {( 1 + {‖ {y}_{i} - {y}_{j} ‖}^{2} )}^{- 1} ( {y}_{i} - {y}_{j} ) ,$$

wherein, C represents the objective function, yᵢ is a present coordinate of cell i on one axis, and yⱼ is a present coordinate of cell j on the same axis;

with the gradient direction as a coordinate updating direction, the cell coordinates are updated with a fixed step size, and a plurality of iterations are performed.

Further, when a distance between cell i and cell j is smaller than a minimum distance r between two cells in the three-dimensional space, if pᵢⱼ−qᵢⱼ>0, let pᵢⱼ−qᵢⱼ=s, wherein s is a negative number not smaller than −1.

Further, the cell-cell interaction intensity matrix A is obtained according to a public receptor-ligand database based on the single-cell transcriptome sequencing data; every element in the cell-cell interaction intensity matrix A is divided by Zₚ, a sum of all elements in the cell-cell interaction intensity matrix A, to obtain the probability matrix P of the cell-cell interaction intensity matrix A, Zₚ=Σᵢ₌₁^{I}Σⱼ₌₁^{I}Σₖ₌₁^{K}w_{L}_{ₖ}_{,R}_{ₖ}(eᵢ^{L}^{ₖ}×eⱼ^{R}^{ₖ}+eᵢ^{R}^{ₖ}×eⱼ^{L}^{ₖ}) for i≠j, wherein:

I is a total number of cells;

K is a total number of ligand-receptor pairs;

w_{L}_{ₖ}_{,R}_{ₖ} represents a chemical binding constant of ligand-receptor pair k;

eᵢ^{L}^{ₖ} is an expression level of ligand k in cell i;

eᵢ^{R}^{ₖ} is an expression level of receptor k in cell i;

eⱼ^{L}^{ₖ} is an expression level of ligand k in cell j;

eⱼ^{R}^{ₖ} is an expression level of receptor k in cell j;

Further, the elements in the probability matrix P of the cell-cell interaction intensity matrix A are:

$${p}_{ij} = \frac{1}{{Z}_{p}} {\sum }_{k = 1}^{K} {w}_{{L}_{k} , {R}_{k}} ( {e}_{i}^{{L}_{k}} \times {e}_{j}^{{R}_{k}} + {e}_{i}^{{R}_{k}} \times {e}_{j}^{{L}_{k}} ) for i \neq j .$$

Further, each element in the cell-cell interaction intensity matrix A is an interaction intensity between corresponding cell C1 and cell C2; a relation for the interaction intensity is:

A_{C1,C2}∝Σₖ₌₁^{K}w_{A,B}(A_{C1}×B_{C2}+A_{C2}×B_{C1}),

or

A_{C1,C2}∝Σₖ₌₁^{K}w_{A,B}(A_{C1}×B_{C2}),

or

A_{C1,C2}∝Σₖ₌₁^{K}w_{A,B}(A_{C2}×B_{C1}),

wherein, A_{C1,C2} represents the cell-cell interaction intensity between cell C1 and cell C2;

w_{A,B} represents a weight for an interaction between ligand A and receptor B;

A_{C1} and A_{C2} represent expression levels of ligand A in cell C1 and cell C2, respectively;

B_{C1} and B_{C2} represent expression levels of receptor B in cell C1 and cell C2, respectively;

K represents a total number of ligand-receptor pairs;

Further, the intercellular distance threshold where each cell interacts with h cells on average is determined using the following method:

for each cell, the distance to its h-th nearest neighbor cell is calculated, and the median distance value for all cells to their corresponding h-th nearest neighbors is calculated and set as the intercellular distance threshold.

Further, the probability matrix P of the cell-cell interaction intensity matrix A obtained is discretized before reconstructing the three-dimensional spatial structure of cell interactions.

Further, the expression levels of ligands and receptors are measured using TPM, FPKM, CPM, Counts, TP10K or log 2(TPM+1).

Beneficial effects of the present disclosure: In the method for predicting spatial relations between cells based on single-cell transcriptome sequencing data provided in an embodiment of the present disclosure, it requires only the single-cell transcriptome sequencing data to predict the interactions between cells in the three-dimensional space, which overcomes the limitation that imaging must be performed to obtain the spatial relations of cells. The predicted spatial relations between cells can be used to analyze relevant molecular mechanisms, molecular effects, cellular spatial classes, responses of individuals to treatment, or the utility of different treatment methods, etc, for example, to evaluate the statistical significance of cell-type-cell-type interactions according to the reconstructed spatial structure of cells; in scoring methods for ligand-receptor pairs of cell-cell interactions or cell-type-cell-type interactions; to simulate interference experiments such as gene knockout, overexpression, cell adoptive input, cell ablation, etc. on a computer to evaluate the effects of some gene or cell or genes or cells on the spatial structure of cells; to perform cell clustering based on the reconstructed spatial structure of cells; to search for genes related to responses or resistance to cell therapy or immunotherapy by analyzing the differentially expressed genes of cell types defined based on the spatial structure; and to deduce if a patient or type of disease achieves a good or poor response to cell therapy or immunotherapy based on the reconstructed spatial structure information of cells.

### BRIEF DESCRIPTION OF THE DRAWINGS

FIG. 1 is a flowchart illustrating a method for predicting spatial relations between cells based on single-cell transcriptome sequencing data provided in an embodiment of the present disclosure;

FIG. 2 is a flowchart illustrating a method for predicting spatial relations between cells based on single-cell transcriptome sequencing data provided in yet another embodiment of the present disclosure;

FIG. 3 is a flowchart illustrating a method for predicting spatial relations between cells based on single-cell transcriptome sequencing data in an example of the present disclosure;

FIG. 4 is a graph showing the distribution of all cells in the three-dimensional coordinate system after initialization, in a method for predicting spatial relations between cells based on single-cell transcriptome sequencing data provided in an embodiment of the present disclosure; and

FIG. 5 is a schematic diagram showing the updating process of cell coordinates, in a method for predicting spatial relations between cells based on single-cell transcriptome sequencing data provided in an embodiment of the present disclosure.

### DETAILED DESCRIPTION

In order to make the objects, technical scheme and advantages of the present disclosure more apparent, the present disclosure is further described in detail with reference to specific embodiments and drawings. Those skilled in the art will appreciate that the present disclosure is not limited to the drawings and the following embodiments.

The inventors of the present disclosure believe that the cell interactions mediated by ligand-receptor pairs play an important role in the formation of the spatial structure of cells, which is formed by competition for spatial positions between interacting cells. On this basis, an embodiment of the present disclosure provides a method for predicting spatial relations between cells based on single-cell transcriptome sequencing data, which comprises the following steps:

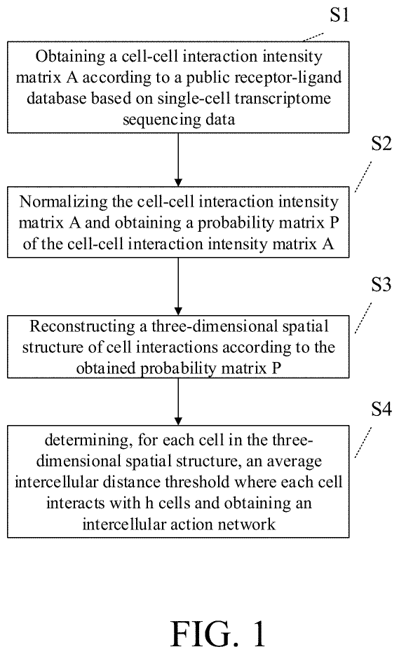

An embodiment of the present disclosure provides a method for predicting spatial relations between cells based on single-cell transcriptome sequencing data, which is characterized in that a cell-cell interaction intensity matrix is calculated according to single-cell transcriptome sequencing data, and a three-dimensional spatial structure of cell interactions is reconstructed according to the cell-cell interaction intensity matrix obtained in the first calculation step. As shown in FIG. 1, the method comprises:

Step S1: obtaining a cell-cell interaction intensity matrix A according to a public receptor-ligand database based on single-cell transcriptome sequencing data;

The cell-cell interaction intensity between two cells can be calculated according to a gene expression matrix E obtained based on single-cell transcriptome sequencing data and a public receptor-ligand database, such as CellphoneDB. A relation of the cell-cell interaction intensity between two cells is expressed according to the law of mass action in chemical reactions as:

A_{C1,C2}∝Σₖ₌₁^{K}w_{A,B}(A_{C1}×B_{C2}+A_{C2}×B_{C1}),

or

A_{C1,C2}∝Σₖ₌₁^{K}w_{A,B}(A_{C1}×B_{C2}),

or

A_{C1,C2}∝Σₖ₌₁^{K}w_{A,B}(A_{C2}×B_{C1}),

wherein, A_{C1,C2} represents the cell-cell interaction intensity between cell C1 and cell C2; w_{A,B} represents a weight for the interaction between ligand A and receptor B; A_{C1} and A_{C2} represent expression levels of ligand A in cell C1 and cell C2, respectively; B_{C1} and B_{C2} represent expression levels of receptor B in cell C1 and cell C2, respectively; K represents a total number of ligand-receptor pairs; The value of w_{A,B} is 1 by default, and can be replaced accordingly depending on the chemical properties or other properties of a ligand-receptor pair.

In this formula, the expression levels of the ligand and receptor can be measured using various methods such as TPM, FPKM, CPM, Counts, TP10K, log 2(TPM+1), etc. For example, when the expression levels are measured using TPM (transcripts per million), the formula for calculating the cell-cell interaction intensity between the two cells described above is given:

A_{C1,C2}∝Σₖ₌₁^{K}w_{A,B}(A_{C1}^{TPM}×B_{C2}^{TPM}+A_{C2}^{TPM}×B_{C1}^{TPM}),

or

A_{C1,C2}∝Σₖ₌₁^{K}w_{A,B}(A_{C1}^{TPM}×B_{C2}^{TPM}),

or

A_{C1,C2}∝Σₖ₌₁^{K}w_{A,B}(A_{C2}^{TPM}×B_{C1}^{TPM}),

In a preferred embodiment of the present disclosure, the A_{C1,C2} calculated above is subjected to a monotonic transformation such as exponential transformation, log transformation, power-law transformation, etc.

After the cell-cell interaction intensities of all the cell pairs are obtained, a cell-cell interaction intensity matrix A can be obtained. Each element in the cell-cell interaction intensity matrix A is an interaction intensity between the corresponding cell C1 and cell C2, and the interaction intensity has the relation described above.

Step S2: normalizing the cell-cell interaction intensity matrix A, and dividing each element in the cell-cell interaction intensity matrix A by Zₚ, a sum of all elements in the cell-cell interaction intensity matrix A, to obtain a probability matrix P of the cell-cell interaction intensity matrix A, with the elements in the probability matrix P being:

$${p}_{ij} = \frac{1}{{Z}_{p}} {\sum }_{k = 1}^{K} {w}_{{L}_{k} , {R}_{k}} ( {e}_{i}^{{L}_{k}} \times {e}_{j}^{{R}_{k}} + {e}_{i}^{{R}_{k}} \times {e}_{j}^{{L}_{k}} ) for i \neq j , \\ {Z}_{p} = {\sum }_{i = 1}^{I} {\sum }_{j = 1}^{I} {\sum }_{k = 1}^{K} {w}_{{L}_{k} , {R}_{k}} ( {e}_{i}^{{L}_{k}} \times {e}_{j}^{{R}_{k}} + {e}_{i}^{{R}_{k}} \times {e}_{j}^{{L}_{k}} ) for i \neq j ,$$

wherein, pᵢⱼ is an interaction intensity between cell i and cell j in the probability matrix P of the cell-cell interaction intensity matrix A;

K is a total number of ligand-receptor pairs;

w_{L}_{ₖ}_{,R}_{ₖ} represents a chemical binding constant of ligand-receptor pair k; its value is 1 by default, or can be experimentally determined;

eᵢ^{L}^{ₖ} is an expression level of ligand k in cell i;

eᵢ^{R}^{ₖ} is an expression level of receptor k in cell i;

eⱼ^{L}^{ₖ} is an expression level of ligand k in cell j;

eⱼ^{R}^{ₖ} is an expression level of receptor k in cell j;

Step S3: reconstructing a three-dimensional spatial structure of cell interactions according to the obtained probability matrix P of the cell-cell interaction intensity matrix A, wherein a model for the reconstructed three-dimensional spatial structure of cell interactions is as follows:

minimizing an objective function

$${\sum }_{i = 1}^{I} {\sum }_{j = 1}^{I} {p}_{ij} log \frac{{p}_{ij}}{{q}_{ij}} , i \neq j ,$$

defined by the Kullback-Leibler divergence, such that:

$${q}_{ij} = \frac{1}{{Z}_{q}} \times \frac{1}{1 + {d}_{ij}^{2}} , for i \neq j , \\ {Z}_{q} = {\sum }_{i = 1}^{I} {\sum }_{j = 1}^{I} \frac{1}{1 + {d}_{ij}^{2}} , i \neq j , \\ {d}_{ij}^{2} = \sqrt{{\sum }_{m = 1}^{3} {( {y}_{i}^{m} - {y}_{j}^{m} )}^{2}} for i \neq j , \\ {d}_{ij} \geq r for i \neq j \\ \vert {y}_{i}^{m} \vert \leq R ,$$

wherein, I is a total number of cells;

qᵢⱼ is a probability of cell j being around cell i;

dᵢⱼ is a Euclidean distance between cell i and cell j in a three-dimensional space;

yᵢ^{m} is a coordinate of cell i on axis m;

yⱼ^{m} is a coordinate of cell j on axis m;

r is a minimum distance between two cells;

R is a radius of the three-dimensional space, and is far greater than r.

In the formula described above, the objective function is defined by the Kullback-Leibler divergence, and pᵢⱼ, qᵢⱼ and dᵢⱼ are defined. The steric hindrance effects are expressed through above inequations.

Step S4: selecting, for each cell in the reconstructed three-dimensional spatial structure of cell interactions, an intercellular distance threshold where each cell interacts on average with h cells so that each cell interacts on average with h cells, and obtaining an intercellular action network.

Specifically, h is the number of cells interacting with the present cell, and can be selected by those skilled in the art as desired; for example, h is 3, 5, 10, etc. For each cell, the distance to the cell closest to it in the hth order is calculated, and the median distance value for all cells is calculated and set as the intercellular distance threshold. After the intercellular distance threshold is obtained, for each pair of cells, if their distance is smaller than the threshold, they are considered to interact with each other; if their distance is greater than the threshold, they are considered to not interact with each other. Thus, a cell interaction network is obtained.

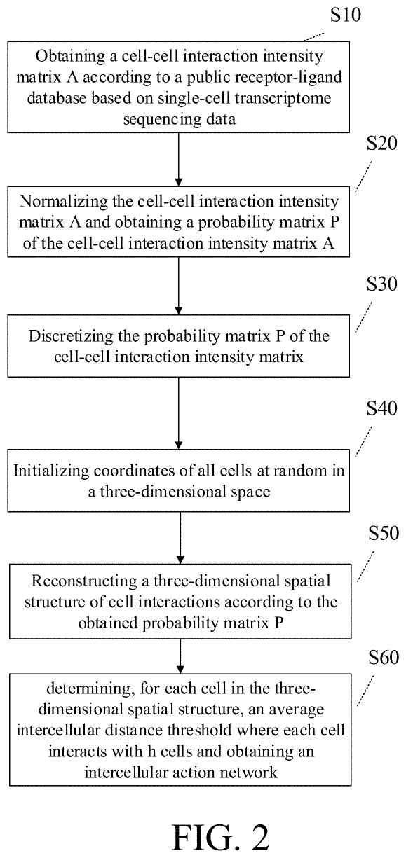

In one specific embodiment of the present disclosure, as shown in FIG. 2, the method for predicting spatial relations between cells based on single-cell transcriptome sequencing data comprises the following steps:

Step S10: obtaining a cell-cell interaction intensity matrix A according to a public receptor-ligand database based on single-cell transcriptome sequencing data.

In an embodiment of the present disclosure, as described above, the expression levels of the ligand and receptor can be measured using TPM. The ligand-receptor TPM value for each single cell is read according to the public receptor-ligand database, and thus the cell-cell interaction intensity matrix A is obtained.

Step S20: normalizing the cell-cell interaction intensity matrix A, and dividing each element in the cell-cell interaction intensity matrix A by Zₚ, a sum of all elements in the cell-cell interaction intensity matrix A, to obtain a probability matrix P of the cell-cell interaction intensity matrix A, with the elements in the probability matrix P being:

$${p}_{ij} = \frac{1}{{Z}_{p}} {\sum }_{k = 1}^{K} {w}_{{L}_{k} , {R}_{k}} ( {e}_{i}^{{L}_{k}} \times {e}_{j}^{{R}_{k}} + {e}_{i}^{{R}_{k}} \times {e}_{j}^{{L}_{k}} ) for i \neq j .$$

Step S30: discretizing the probability matrix P of the cell-cell interaction intensity matrix.

In a preferred embodiment of the present disclosure, the probability matrix P of the cell-cell interaction intensity matrix is discretized. It is usually sufficient to select the largest first 50 elements in each row or column.

Those skilled in the art will appreciate that this step is optional, and it is feasible to not include this step.

Step S40: initializing the coordinates of all cells in a three-dimensional space at random.

In a three-dimensional space, the position of a random cell is used as an origin, and the coordinates of other cells are determined.

Step S50: reconstructing a three-dimensional spatial structure of cell interactions according to the obtained probability matrix P of the cell-cell interaction intensity matrix A, wherein a model for the reconstructed three-dimensional spatial structure of cell interactions is as follows:

minimizing an objective function

$${\sum }_{i = 1}^{I} {\sum }_{j = 1}^{I} {p}_{ij} log \frac{{p}_{ij}}{{q}_{ij}} , i \neq j .$$

Step S60: selecting, for each cell in the reconstructed three-dimensional spatial structure of cell interactions, an intercellular distance threshold where each cell interacts on average with h cells so that each cell interacts on average with h cells, and obtaining an intercellular action network.

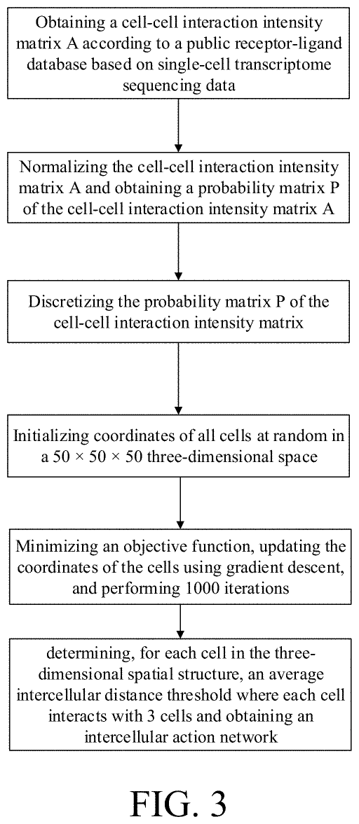

By way of example, the method for predicting spatial relations between cells of the present disclosure is illustrated below using single-cell transcriptome data of 5000 cells in the melanoma database, as shown in FIG. 3.

Based on the single-cell transcriptome sequencing data, a cell-cell interaction intensity matrix A is obtained according to a public receptor-ligand database, and a probability matrix P of the cell-cell interaction intensity matrix A is further obtained. In an embodiment of the present disclosure, the expression levels of the ligand and receptor can be measured using TPM.

The probability matrix P of the cell-cell interaction intensity matrix is discretized, and the largest 50 elements in each row of the matrix are kept.

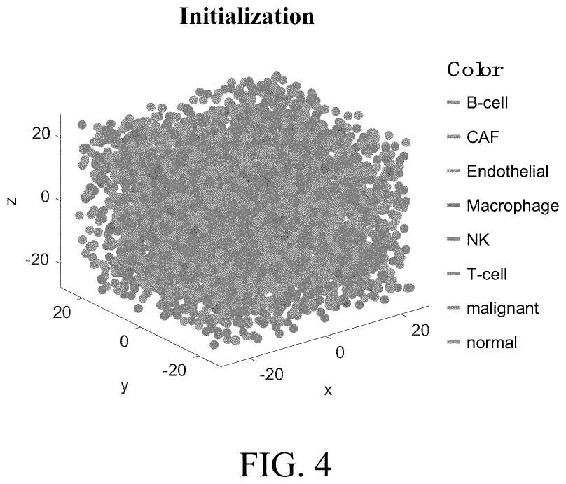

In a 50×50×50 three-dimensional space, the coordinates of all cells are initialized at random. In the case of the melanoma database of this embodiment, all the cells after the initialization distribute in the three-dimensional coordinate system as shown in FIG. 4, wherein B-cell represents B cells, CAF represents cancer-associated fibroblasts, Endothelial represents endothelial cells, Macrophage represents macrophages, NK represents natural killer cells, T-cell represents T cells, Malignant represents tumor cells, and Normal represents normal cells.

The objective function

$${\sum }_{i = 1}^{I} {\sum }_{j = 1}^{I} {p}_{ij} log \frac{{p}_{ij}}{{q}_{ij}} , i \neq j$$

is minimized, and the cell coordinates are updated using gradient descent.

A gradient direction is calculated for each cell at the present coordinates:

$$\frac{δ C}{δ {y}_{i}} = 4 {\sum }_{j} ( {p}_{ij} - {q}_{ij} ) {( 1 + {‖ {y}_{i} - {y}_{j} ‖}^{2} )}^{- 1} ( {y}_{i} - {y}_{j} ) ,$$

wherein, C represents the objective function, yᵢ is a present coordinate of cell i on one axis , and yⱼ is a present coordinate of cell j on the same axis. With the gradient direction as a coordinate updating direction, the cell coordinates are updated with a fixed step size, and a plurality of iterations are performed. A total of 1000-2000 iterations are performed. In this embodiment, 1000 iterations are performed.

In view of the steric hindrance effects,

dᵢⱼ≥r for i≠j,

|yᵢ^{m}|≤R,

In the present embodiment, r=0.01, and R=50. When the distance between cell i and cell j is smaller than r=0.01, if pᵢⱼ−qᵢⱼ>0 in the formula described above, let pᵢⱼ−qᵢⱼ=s, wherein s is a negative number not smaller than −1. When there are cells with the coordinates greater than R=50, the coordinates of all the cells are scaled down equally so that the coordinates of all the cells are still smaller than R=50.

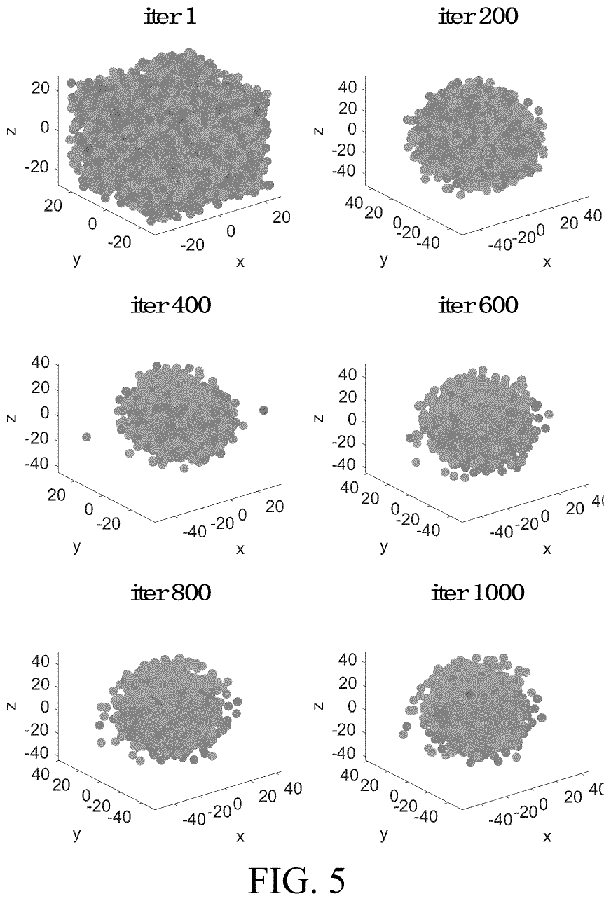

The process of updating the coordinates of the cells in this step is shown in FIG. 5. Schematic diagrams of the cells in the three-dimensional coordinate system after 200, 400, 600, 800 and 1000 iterations are shown in FIG. 5.

For each cell in the reconstructed three-dimensional spatial structure of cell interactions, an intercellular distance threshold where each cell interacts on average with 3 cells is selected so that each cell interacts on average with 3 cells, and an intercellular action network is obtained.

In the specification, description involving the term “one embodiment”, “some embodiments”, “examples”, “a specific example”, “some examples” or the like means that a particular feature, structure, material or characteristic described in reference to the embodiment or example is included in at least one embodiment or example of the present disclosure. In this specification, the schematic descriptions of the terms described above do not necessarily refer to the same embodiment or example. Moreover, the specific features, materials, structures and other characteristics described may be combined in any one or more embodiments or examples in an appropriate manner.

Examples of the present disclosure have been described above. However, the present disclosure is not limited thereto. Any modification, equivalent, improvement and the like made without departing from the spirit and principle of the present disclosure shall fall within the protection scope of the present disclosure.

1. A method for predicting spatial relations between cells based on single-cell transcriptome sequencing data, comprising:
acquiring a probability matrix P of a cell-cell interaction intensity matrix A based on single-cell transcriptome sequencing data;
reconstructing a one/two/three-dimensional spatial structure of cell interactions according to the acquired probability matrix P of the cell-cell interaction intensity matrix A; and obtaining an intercellular action network from the reconstructed three-dimensional spatial structure by setting a threshold distance estimated by the average number of neighbor cells around one cell.

2. The method according to claim 1, wherein a model for reconstructing a three-dimensional spatial structure of cell interactions is as follows:
minimizing an objective function
$${\sum }_{i = 1}^{I} {\sum }_{j = 1}^{I} {p}_{ij} log \frac{{p}_{ij}}{{q}_{ij}} , i \neq j ,$$
such that:
$${q}_{ij} = \frac{1}{{Z}_{q}} \times \frac{1}{1 + {d}_{ij}^{2}} , for i \neq j , \\ {d}_{ij}^{2} = \sqrt{{\sum }_{m = 1}^{3} {( {y}_{i}^{m} - {y}_{j}^{m} )}^{2}} for i \neq j , \\ {Z}_{q} = {\sum }_{i = 1}^{I} {\sum }_{j = 1}^{I} \frac{1}{1 + {d}_{ij}^{2}} , i \neq j ,$$
wherein, I is a total number of cells;
pᵢⱼ is an interaction intensity between cell i and cell j in the probability matrix P of the cell-cell interaction intensity matrix A;
qᵢⱼ is a probability of cell j being around cell i;
dᵢⱼ is a Euclidean distance between cell i and cell j in a three-dimensional space;
yᵢ^{m} is a coordinate of cell i on axis m;
yⱼ^{m} is a coordinate of cell j on axis m;

3. The method according to claim 2, wherein the objective function
$${\sum }_{i = 1}^{I} {\sum }_{j = 1}^{I} {p}_{ij} log \frac{{p}_{ij}}{{q}_{ij}} , i \neq j$$
is minimized, cell coordinates are updated using gradient descent, and a gradient direction is calculated for each cell at the present coordinates:
$$\frac{δ C}{{δ y}_{i}} = 4 {\sum }_{j} ( {p}_{ij} - {q}_{ij} ) {( 1 + {‖ {y}_{i} - {y}_{j} ‖}^{2} )}^{- 1} ( {y}_{i} - {y}_{j} ) ,$$
wherein, C represents the objective function, yᵢ is a present coordinate of cell i on one axis, and yⱼ is a present coordinate of cell j on the same axis;
with the gradient direction as a coordinate updating direction, the cell coordinates are updated with a fixed step size, and a plurality of iterations are performed.

4. The method according to claim 3, wherein when a distance between cell i and cell j is smaller than a minimum distance r between two cells in the three-dimensional space, if pᵢⱼ−qᵢⱼ>0, let pᵢⱼ−qᵢⱼ=s, wherein s is a negative number not smaller than −1.

5. The method according to claim 1, wherein the cell-cell interaction intensity matrix A is obtained according to a public receptor-ligand database based on the single-cell transcriptome sequencing data; every element in the cell-cell interaction intensity matrix A is divided by Zₚ, a sum of all elements in the cell-cell interaction intensity matrix A, to obtain the probability matrix P of the cell-cell interaction intensity matrix A,
Zₚ=Σᵢ₌₁^{I}Σⱼ₌₁^{I}Σₖ₌₁^{K}w_{L}_{ₖ}_{,R}_{ₖ}(eᵢ^{L}^{ₖ}×eⱼ^{R}^{ₖ}+eᵢ^{R}^{ₖ}×eⱼ^{L}^{ₖ}) for i≠j, wherein:
I is a total number of cells;
K is a total number of ligand-receptor pairs;
w_{L}_{ₖ}_{,R}_{ₖ} represents a chemical binding constant of ligand-receptor pair k;
eᵢ^{L}^{ₖ} is an expression level of ligand k in cell i;
eᵢ^{R}^{ₖ} is an expression level of receptor k in cell i;
eⱼ^{L}^{ₖ} is an expression level of ligand k in cell j;
eⱼ^{R}^{ₖ} is an expression level of receptor k in cell j.

6. The method according to claim 5, wherein the elements in the probability matrix P of the cell-cell interaction intensity matrix A are:
$${p}_{ij} = \frac{1}{{Z}_{p}} {\sum }_{k = 1}^{K} {w}_{{L}_{k} , {R}_{k}} ( {e}_{i}^{{L}_{k}} \times {e}_{j}^{{R}_{k}} + {e}_{i}^{{R}_{k}} \times {e}_{j}^{{L}_{k}} ) for i \neq j ,$$

7. The method according to claim 1, wherein each element in the cell-cell interaction intensity matrix A is an interaction intensity between corresponding cell C1 and cell C2; a relation for the interaction intensity is:
A_{C1,C2}∝Σₖ₌₁^{K}w_{A,B}(A_{C1}×B_{C2}+A_{C2}×B_{C1}),
or
A_{C1,C2}∝Σₖ₌₁^{K}w_{A,B}(A_{C1}×B_{C2}),
or
A_{C1,C2}∝Σₖ₌₁^{K}w_{A,B}(A_{C2}×B_{C1}),
wherein, A_{C1,C2} represents the cell-cell interaction intensity between cell C1 and cell C2;
w_{A,B} represents a weight for an interaction between ligand A and receptor B;
A_{C1} and A_{C2} represent expression levels of ligand A in cell C1 and cell C2, respectively;
B_{C1} and B_{C2} represent expression levels of receptor B in cell C1 and cell C2, respectively;
K represents a total number of ligand-receptor pairs.

8. The method according to claim 1, wherein the intercellular distance threshold where each cell interacts on average with h cells is determined using the following method:
for each cell, the distance to the cell closest to it in the hth order is calculated, and the median distance value for all cells is calculated and set as the intercellular distance threshold.

9. The method according to claim 1, wherein the probability matrix P of the cell-cell interaction intensity matrix A obtained is discretized before reconstructing the three-dimensional spatial structure of cell interactions.

10. The method according to claim 5, wherein the expression levels of ligands and receptors are measured using TPM, FPKM, CPM, Counts, TP10K or log 2(TPM+1).

11. The method according to claim 7, wherein the expression levels of ligands and receptors are measured using TPM, FPKM, CPM, Counts, TP10K or log 2(TPM+1).
